(19) United States
(12) Patent Application Publication
(10) Pub. No.: US 2013/0006297 A1
(43) Pub. Date: Jan. 3, 2013

(54) Large Vessel Closure Sheath

(76) Inventor: William Joseph Drasler, Minnetonka, MN (US)

(21) Appl. No.: 13/539,023

(22) Filed: Jun. 29, 2012

Related U.S. Application Data
(60) Provisional application No. 61/571,555, filed on Jun. 30, 2011.

(51) Int. Cl. A61B 17/08

(52) U.S. Cl. 606/213

(57) ABSTRACT

A vascular closure device for closing vessels of the body following percutaneous access via an introducer sheath that is similar in size to the vessel lumen. The vascular closure device has an anchor that is inflated using a polymerizable polymer. The anchor is attached to a plug that also can be inflated with a polymer. The anchor and plug can be introduced into the body via a positionable introducer sheath that is also used for the therapeutic procedure. A weep hole in the introducer sheath is positionable adjacent the arteriotomy site.

### CROSS REFERENCE TO RELATED APPLICATIONS

This patent application makes reference to and includes all information found in the provisional patent application No. 61/571,555 entitled Large Vessel Closure Sheath, filed 30 Jun. 2012 by William J. Drasler, et. al.

### FIELD OF THE INVENTION

This invention relates to a medical device that is used to provide closure to an access site in a blood vessel or other vessel of the body following an interventional procedure. It is further related to closing a large opening in an artery following an interventional procedure using a large diameter catheter relative to the blood vessel diameter. It has significant relevance to closing an access site opening in the femoral artery following percutaneous aortic valve implantation or delivery of other large profile interventional devices.

### BACKGROUND OF THE INVENTION

Many vascular closure devices are well suited to closing an access site in the femoral artery following interventional coronary angioplasty, coronary stenting, peripheral interventions in the leg, and other interventional procedures where typically 6-8 French introducer sheaths are used to provide passage for the interventional catheter. When larger interventional catheter devices are used, for example, to perform a transcutaneous aortic valve implantation (TAVI) procedure, often a surgical cutdown is required to expose the femoral artery and a surgical vessel closure is performed to establish vessel closure. Often an 18-21 French introducer sheath can be required to provide passage for the large 18-21 French interventional catheters to pass through the vascular access site in a femoral artery that is often 7-8 mm in diameter; this is not much larger than the introducer sheath and can sometimes be smaller in diameter than the introducer sheath.

Several issues contribute to the difficulty for existing vascular closure devices to be used consistently for closure of large access site openings, particularly for those devices that utilize an anchor located within the blood vessel lumen (250) and a plug located on the outside of the blood vessel wall (150). To ensure that the anchor is released into the blood vessel at a location near the access site, the working introducer sheath is replaced by a replacement introducer sheath that has a bleed hole to determine its location within the blood vessel. This replacement works well for smaller, 6 French, introducer sheaths but is not well tolerated for larger access site openings due to the potential for significant bleeding.

For small access site openings, a soft degradable plug material is delivered to the outside of the blood vessel wall (150) to provide the primary hemostasis element. This plug is held in place by an attachment to the anchor located within the vessel lumen. For large vessel access site closure, the opening in the blood vessel is similar in size or larger than the plugs currently being used and the current plug configurations can provide a significant potential for embolization of the plug into the blood vessel. Existing anchor and plug systems for vascular closure are thus not reliable and consistent for closure of large access site openings currently created for the delivery of TAVI and other large interventional catheter devices.

What is needed is a closure system that can reliably and consistently close a large 16-22 French opening in a femoral artery following the delivery of a large interventional device.

### SUMMARY

The present invention is well suited to use as a closure device for closure of an access site following a TAVI procedure or other percutaneous procedure that requires a large interventional catheter and is not easily sealed using standard manual compression via thumb pressure or the current percutaneous closure devices. The present invention overcomes many of the problems that occur when using a closure device having a luminal anchor and an external plug to seal a large diameter access site opening. These problems include the lack of space for an anchor-type sealing device to be deployed at the end of a delivery sheath when the sheath is approximately the same size as the lumen of the blood vessel.

The present closure device comprises the working introducer sheath normally used for delivery of the therapeutic catheter as a portion of the closure system. Following the completion of the therapeutic procedure the therapeutic interventional catheter is removed from the working introducer sheath and the working introducer sheath is repositioned. In one embodiment the distal end of the introducer sheath is located within approximately 8 mm or less of the access site opening within the blood vessel lumen (250). To accomplish this, the working introducer sheath is formed with an axial passage that extends throughout the wall of the catheter from a weep hole located within the blood vessel lumen (250) to an opening in the proximal manifold. This axial passage is maintained in a patent condition by inserting a mandrel into the passage while the catheter is in use. Alternately, fluid such as heparinized saline can be injected into the passage to ensure that is maintained patent during the procedure. In another embodiment the introducer sheath can be introduced into the blood vessel for a distance greater than 8 mm when the anchor is being deployed; this allows a greater safety margin to ensure that the introducer sheath is not inadvertently placed outside of the access site before the anchor has been properly deployed into the vessel lumen.

Within the central lumen of the introducer sheath an inner sheath is placed that contains an anchor, a plug, and a connecting fiber that joins the anchor with the plug. The connecting fiber can be hollow and can be used to deliver a fluid if desired to the anchor or the plug. The anchor is an elongated member that is located within the lumen of the blood vessel. It is anticipated that the anchor is biodegradable and is formed from a material such as polyglycolic acid (PGA), polylactic acid (PLA), copolymers or combinations of PGA and PLA, tyrosine polycarbonate (TPC) polymer, or other biodegradable polymer or metal. The plug is formed from a biodegradable material that can also include PGA, PLA, TPC, along with a number of additional more compressible biodegradable materials in porous forms or as in foams including collagen, gelatin, polyethylene glycol (PEG), and various clot-forming materials including starches and other biodegradable materials used in the medical device industry. The plug is delivered in a small diameter configuration and expands out upon contact with blood or via a delivery of inflation fluid to form a configuration that is larger than the arteriotomy site diameter. The fiber can be a biodegradable fiber such as that found in suture materials including PGA, PLA, polycaprolactone and others. The fiber in one embodiment can be adjustable in length to allow the plug to be pushed along the fiber and into contact with the outside of the blood vessel wall (150). In an alternate embodiment, the fiber can have a fixed length to set the distance between the plug and the anchor such that it is approximately equal to the thickness of the blood vessel wall (150); the fiber of this embodiment can be elastic to allow some stretch between the plug and the anchor during deployment.

In one embodiment, a portion of the plug consists of a more elastic component or frame that is able to expand out upon release from inner sheath to form a diameter that is larger than the diameter of the access site opening in the blood vessel wall (150). This frame ensures that the plug and the anchor cannot embolize into the blood vessel. Another portion of the plug is formed from a more compressible biodegradable material to provide the hemostatic function of the plug.

In an embodiment having a plug that has both a frame component and a softer or more flexible portion or component, the frame can be attached to the anchor by a connecting fiber that has a fixed length. The fixed length can be similar to the wall thickness of the blood vessel although this length can be varied according to the volume of the plug material. The length can be insignificant in length or it can be elastic and expand in length. Alternately, the frame can be moved slidably along the connecting fiber such that the plug, including the frame, can be pulled snugly against the outside of the vessel wall. A cinch ring (135) or a knot placed along the connecting fiber can be used to hold the plug tightly against the vessel wall.

In an alternate embodiment the plug is formed of entirely soft compressible biodegradable material. This plug material can be compressed by sliding a friction fit cinch ring (135) along the fiber to push the plug into contact with the outside of the blood vessel wall (150). To help direct the plug material to its position adjacent to the blood vessel, an expandable plug container can be located around the outer perimeter of the plug. The shape of the expandable plug container can be tapered outwardly near the blood vessel wall (150) to allow a greater amount of plug material to be deposited adjacent the outside of the blood vessel. This increased amount of plug material adjacent the access site opening will reduce the ability of the plug material from embolizing into the lumen of the blood vessel and will enhance the hemostatic seal. Axially directed wires can be attached to the expandable plug container to ensure that plug material is directed properly to the outer surface of the blood vessel.

The method of delivery of the present vascular occlusion device can require fewer steps than with existing plug and anchor vascular closure devices. The present invention obviates the need for exchange of the working introducer sheath for a new external sheath as found in existing closure devices. The use of a plug having a portion that has a biodegradable elastic frame not only ensures safety against embolization of the plug but also allows the delivery of the plug of one embodiment to occur automatically by direct removal of the inner sheath of the present device.

During delivery of the anchor of one embodiment of the invention to the lumen of the vessel, the anchor can be released while the introducer sheath is inserted more than approximately 8 mm into the vessel to ensure that the introducer sheath is not inadvertently pulled out of the vessel prematurely prior to adequate release of the anchor. Alternately, the anchor can be released after the distal end of the introducer is retracted to within approximately 3-8 mm of the access site such that more space is made available between the distal end of the introducer sheath and the vessel wall for delivery of the anchor to the lumen of the vessel with the introducer sheath partially retracted.

To ensure that the biodegradable frame retains its final configuration at a diameter that is larger than the access site opening in the blood vessel, the frame is stored in a relaxed state similar to its fully expanded state. The frame is then loaded into the inner sheath just prior to use and expands outwards upon release from the inner sheath. Storage of the frame in a relaxed state ensures that the frame does not creep over time and upon exposure to temperature or humidity that can be present during the sterilization or storage of the device.

It is understood that the device of the present invention is not limited only to vascular closure of large access site openings and can be used in any vascular closure application. When used for standard 6 French vascular access site closure, the dimensions of the introducer catheter and other elements can be altered proportionally and accordingly.

Several embodiments for the anchor are presented; an elongated anchor can be formed from a biodegradable material as described earlier; alternately, the anchor can be formed from a wire or fiber that expands outward upon delivery into the vessel lumen to a dimension that is larger than the arteriotomy site diameter. The wire or fiber that has been folded or coiled and can be a metal such as Nitinol, stainless steel, or other elastic or nonelastic metal used for medical device implants; it can be a polymeric material such as Dacron, polyethylene, or a biodegradable wire. A cover material can be attached to the wire or fiber anchor to provide additional hemostasis character to the device at the arteriotomy site.

Alternately, the anchor can be formed from a polymeric bag with a flat disc shape. The anchor bag can be filled with a fluid including a polymeric fluid or gel that polymerizes after the anchor bag has been delivered to the lumen of the blood vessel. The flat anchor can form a well-fitting cap over the arteriotomy site and conform to the shape of the vessel wall on the luminal side before the polymer hardens to a solid. The anchor bag and its contents can be made from biodegradable materials or can be formed from nonbiodegradable polymeric or composite materials. The anchor is delivered in a small diameter configuration that is able to fit within the lumen of an introducer sheath or inner sheath and is expanded out to a larger diameter than the arteriotomy site after it has been delivered to the blood vessel lumen (250). The plug can similarly be formed from a polymeric bag that contains a polymeric fluid or gel that becomes harder or polymerizes after delivery to the outside surface of the blood vessel wall and inside the tissue tract in the subcutaneous tissue. The plug is delivered via an introducer sheath or inner sheath in a small diameter configuration that is smaller than the arteriotomy site and expands out to form a deployed configuration that is larger than the arteriotomy site. The polymer or gel used in the anchor bag or plug bag can solidify in a period of time ranging from 5 minutes to 2 hours.

### BRIEF DESCRIPTION OF THE DRAWINGS

FIG. 1A is a plan view of an introducer sheath having a weep hole.

FIG. 1B is a cross sectional view of the introducer sheath.

FIG. 2A is a plan view of the distal end of one embodiment of the introducer sheath.

FIG. 2B is a plan view of the distal end of an alternate embodiment of the introducer sheath.

FIG. 2C is a plan view of the distal end of an introducer sheath having side extensions.

FIG. 3A is a plan view of a vascular closure device with an anchor, a plug and a connecting fiber.

FIG. 3B is a plan view showing a vascular closure device deployed on each side of a blood vessel wall.

FIG. 4A is a plan view of a vascular closure device embodiment with a fixed connecting fiber.

FIG. 4B is a plan view of the device of FIG. 4A in a deployed configuration.

FIG. 5A is a plan view of a plug frame and upper soft portion.

FIG. 5B is a side view of the device of FIG. 5A.

FIG. 5C is a plan view of a vascular closure device embodiment with a fixed connection and inside the inner sheath.

FIG. 5D is a plan view of the device of FIG. 5C deployed on each side of a blood vessel wall.

FIG. 5E is a plan view of a vascular closure device embodiment having a plug frame with a pivotal connection and a frame lock and fixed connecting fiber.

FIG. 5F is a plan view of the embodiment of FIG. 5E deployed across a blood vessel wall.

FIG. 5G is a plan view of the plug frame with a pivotal connection and spokes.

FIG. 5H is a plan view of an embodiment of a vascular closure device with a slidable connection fiber.

FIG. 5I is a plan view of the embodiment of FIG. 5H in a deployed configuration across a blood vessel wall.

FIG. 6 is a plan view of vascular closure device with a soft plug portion.

FIG. 7A is a plan view showing an expandable plug container around the soft plug portion.

FIG. 7B is a cross sectional view through the inner sheath, expandable plug container, and the plug.

FIG. 7C is a plan view showing the plug expanding out to a larger diameter nearest to the anchor.

FIG. 7D is a plan view showing the funnel shape for the expandable container and placing the plug into a larger diameter configuration adjacent the blood vessel wall.

FIG. 8A is a plan view showing a plug comprised of several plug segments each contained by a separate connecting filament.

FIG. 8B is a plan view of the embodiment of FIG. 8A after the plug segments have been pushed into contact with the vessel wall and held by a cinch ring.

FIG. 9 shows an anchor having connecting fibers contained within it to provide addition strength to the anchor.

FIG. 10A is a plan view showing a funnel to assist in loading the plug frame into the inner sheath or into the introducer sheath for delivery to the arteriotomy site.

FIG. 10B is a plan view of the device of FIG. 10A after it has been advanced into the inner sheath or the introducer sheath.

FIGS. 11A-11G show the steps of placing the vascular closure device into an introducer sheath having a weep hole and advancing a vascular closure device across the vessel wall and leaving it in a deployed configuration.

FIG. 12A is a plan view showing the vascular closure device being advanced along with an inner sheath into an introducer sheath into the blood vessel.

FIG. 12B is a plan view of the device of FIG. 12A with the vascular closure device advanced out of the inner sheath and into the blood vessel.

FIG. 12C is a plan view of the device of FIG. 12B with the anchor pulled into contact with the vessel wall.

FIG. 13A is a plan view of an introducer sheath and dilator having a weep hole in the introducer sheath and a passage extending through the dilator.

FIG. 13B is a plan view of an introducer sheath and dilator with a small annular space and a separate lumen extending along the length of the dilator.

FIG. 14A is a plan view of a vascular closure device having an anchor bag that is inflated with inflation medium that polymerized to form a solid and a means for filling the anchor bag.

FIG. 14B is an end view of the anchor bag of FIG. 14A showing the holding fibers.

FIG. 14C is a cross sectional view of the anchor stem of FIG. 14A showing the ridges, slots, and polymer path.

FIG. 14D is a plan view of a vascular closure device having a fillable anchor bag and a fill tube with a valve to prevent polymer flow out of the fill tube.

FIG. 15 is a plan view of the anchor bag showing the holding fibers, the anchor stem, and fill tube used to fill the anchor bag with polymerizable polymer.

FIG. 16A is a plan view showing the inflatable anchor bag being introduced to the vessel lumen via an introducer sheath or an inner sheath.

FIG. 16B shows a plan view of the inflated anchor extending out of the introducer sheath within the blood vessel lumen.

FIG. 16C shows a plan view of the inflated anchor bag pulled against the arteriotomy site.

FIG. 16D shows a plan view of the inflated anchor bag with the fill tube extending proximally and a plug being held against the outside of the vessel wall via a cinch ring.

FIG. 17 is a plan view of an inflatable plug that can be filled with polymerizable polymer to form a more ridged plug after polymerization.

FIG. 18A is a plan view of a vascular closure device comprised of a one-piece anchor and plug fillable with inflation fluid that can harden or polymerize after it has been delivered to the arteriotomy site.

FIG. 18B is an embodiment of the fillable plug and anchor showing one structure for detaching the fill tube from the vascular closure device.

FIGS. 19A-D show the vascular closure device being delivered to the blood vessel lumen and deployed across the vessel wall.

FIG. 20 is a plan view of an anchor having a frame ring and struts covered by an anchor cover.

### DETAILED DESCRIPTION OF THE INVENTION

The present invention is a vascular closure system particularly well suited for closure of large diameter vascular access site openings such as those created during the delivery of interventional devices such as the TAVI device or abdominal aortic aneurysm (AAA) devices. The introducer sheath typically used for these procedures can range from 16 French to over 20 French. A variety of vascular closure devices are currently used for closure of small access site openings such as the 6 French openings typically formed in the femoral artery of the leg for coronary therapeutic procedures. These devices do not work consistently for closure of large access site openings. One device that has not been successful for use in closure of large access site openings has an anchor (130) located within the blood vessel lumen that is attached to a plug (100) located on the outside of the blood vessel. One problem with this current system when used for large access site closure is associated with the exchange of the working introducer sheath for another sheath that is provided as part of the existing vascular closure systems. Another problem is associated with the size of the vascular access site opening and the inability to ensure that the plug (100) does not embolize into the blood vessel. Another problem is inadequate space between the sheath and the vessel lumen to deploy an anchor (130) large enough to ensure that it cannot be pulled out of the vessel through the arteriotomy site.

The present invention provides a working introducer sheath (5) for the interventional procedure that can also be used as one element of the vascular closure device (90). One embodiment of the introducer sheath (5) of the present invention is shown in FIG. 1A having a standard circular opening (17) at its distal end (10). It is introduced into the vasculature over a standard tapered dilator (15) which is advanced into the blood vessel over a guidewire which passes through the guidewire opening (20) and through the guidewire lumen of the dilator (15) using a standard Seldinger access technique. The opening (17) in the distal end (10) of the introducer sheath (5) provides passage for the dilator (15) and also provides passage for the therapeutic catheter. The introducer sheath (5) of the present invention has a wall passage (25) that extends through the wall (30) of the introducer sheath (5) from the manifold (35) to the weep hole (40) located near the distal end (10) of the introducer sheath (5). The weep hole (40) provides a blood flow from the blood vessel lumen (250) to the manifold (35) and notifies the operator that the introducer weep hole (40) is located within the vessel lumen. Use of the weep hole (40) allows the operator to position the distal end (10) of the introducer sheath (5) at a generally small distance of less than one or two centimeters from the blood vessel wall access site to ensure that the introducer sheath (5) is not accidentally pulled out of the blood vessel. The distal end (10) of the introducer sheath (5) can be positioned even closer to the access site such that its distal end (10) is within approximately 8 mm or less, thereby allowing the anchor (130) to be delivered between the distal end (10) of the introducer sheath (5) and the blood vessel wall (150) that is 180 degrees opposed to the access site.

The introducer sheath (5) would typically have one wall passage (25) although it is within the bounds of the invention to have more than one wall passage (25). FIG. 1A shows two wall passages (25) for convenience to allow a description of two different devices and methods for maintaining the wall passage (25) in a patent condition. The wall passage (25) can be an axial wall passage (25) that ends in a weep hole (40) located in the tapered region (45) of the introducer sheath (5). Alternately the wall passage (25) can be a curved passage (50) that curves near the distal end (10) of the introducer sheath (5) to form a weep hole (40) on the outer surface (55) of the introducer sheath (5) near the distal end (10).

A mandrel (60) can extend through the wall passage (25) from the manifold (35) to the weep hole (40) during the interventional therapeutic procedure to ensure that the wall passage (25) does not fill with blood elements that can cause thrombosis of the wall passage (25). The mandrel (60) can be a metal or plastic wire or strand that can be introduced from the manifold (35) and removed from the wall passage (25) by applying a tension force. The diameter of the mandrel (60) can range from 0.002-0.020 inches. Alternately, the wall passage (25) can be filled with heparinized saline (or other antithrombotic agent) and flushed with heparinized saline through a manifold port (65) during the therapeutic interventional procedure to maintain patency of the wall passage (25).

A cross section of introducer sheath (5) having a single wall passage (25) is shown in FIG. 1B with a mandrel (60) contained. The diameter of the wall passage (25) can range from approximately 0.003-0.020 inch to ensure that blood is able to flow through the wall passage (25) and provide necessary input to the operator that the weep hole (40) is located within the blood vessel lumen. The perimeter of the introducer sheath (5) due to the presence of the wall passage (25) has a minimal effect on the overall size of opening made in the blood vessel wall (150) at the access site.

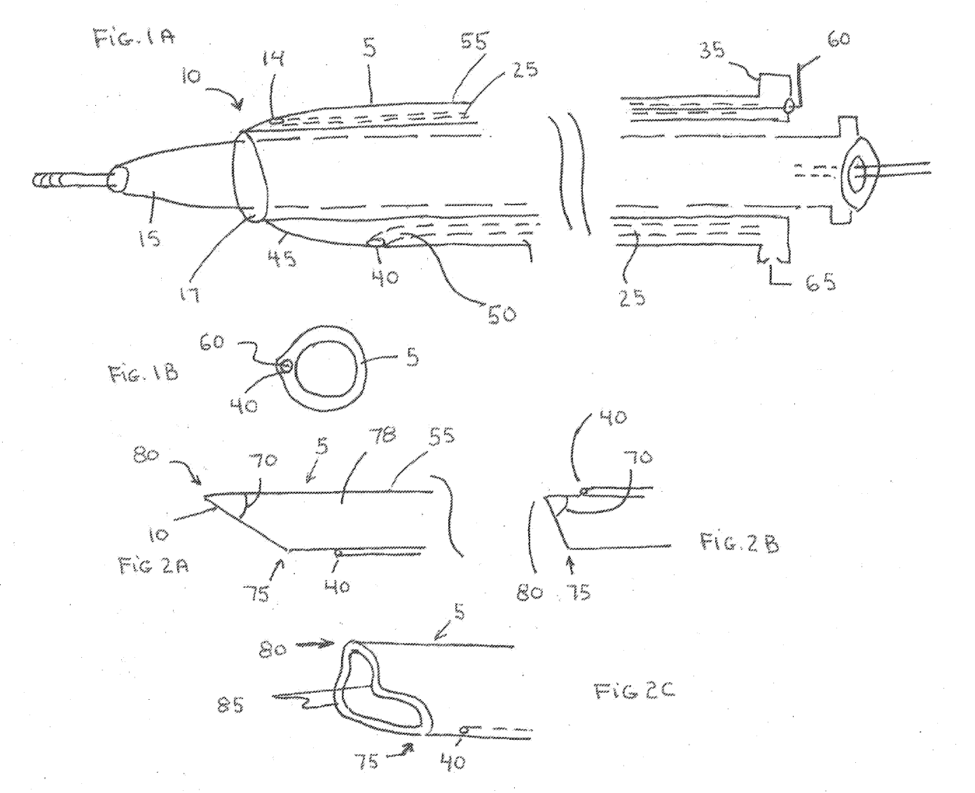

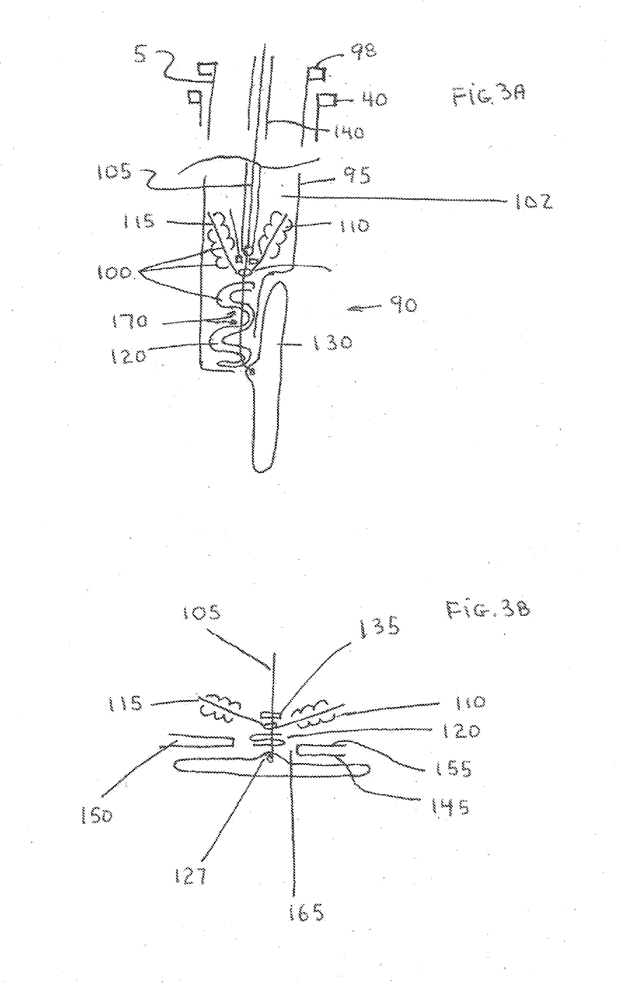

FIGS. 2A-2C show alternate embodiments for the introducer sheath (5) of the present invention. FIG. 2A shows an introducer sheath (5) with a beveled distal end (10) with a bevel angle (70) of approximately 30 degrees; the weep hole (40) for this introducer sheath (5) is located at the heel (75). An open central lumen (78) extends through the introducer sheath (5). Alternately, the introducer sheath (5) can have a bevel angle (70) of approximately 60 degrees as shown in FIG. 2B; this introducer sheath (5) can have a weep hole (40) located at the toe (80). As another alternate embodiment for the introducer sheath (5), the bevel angle (70) can be approximately 45 degrees and the weep hole (40) can be located at either the heel (75) as shown or at the toe (80) or other location along the outer surface (55). An additional embodiment for the introducer sheath (5) has side extensions (85) as shown in FIG. 2D; the side extensions (85) ensure that the introducer sheath (5) is extending significantly into the access site of the blood vessel while the weep hole (40) may not be extending as far into the lumen of the blood vessel. The side extensions (85) do not affect the ability of an anchor (130) to extend in the axial direction of the blood vessel between the distal end (10) of the introducer sheath (5) and the opposite wall of the blood vessel while the side extensions (85) of the introducer sheath (5) are extending at least approximately 1-4 millimeters into the blood vessel.

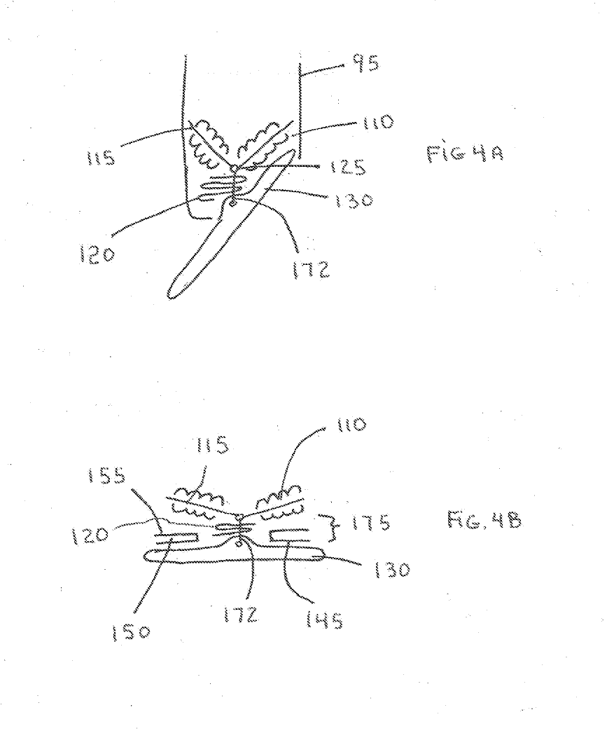

FIGS. 3A and 3B show elements of the vascular closure device (90) of the present invention contained in the inner sheath (95) extending within the outer sheath and having an inner sheath manifold (98) that is located proximal to the outer sheath manifold (35) and as delivered to the blood vessel wall (150). FIG. 3A shows the inner sheath containing a plug (100) located within the inner sheath central corridor (102) that has an adjustable connecting fiber (105) extending through a portion of the plug (100). The vascular closure device (90) could similarly have been placed and delivered to the vasculature in an introducer sheath (5) such as that shown in FIGS. 1A-2C. The plug (100) can have several components, an upper soft plug portion (110) and a more elastic plug frame (115) which can be attached to the upper soft plug portion (110); alternately, the plug (100) can contain only a lower soft plug portion (120) although the lower soft portion can be made up of more than one material and can itself be comprised of more than one portion. The soft plug portion (110 and 120) can have either or both an upper and lower soft plug portion (120). The frame is generally formed from a less compliant or noncompliant material such as PLLA or PGA, for example, or from a metal such as Nitinol or stainless steel. The soft plug portions are formed from compliant materials that are compliant and can deform into an arteriotomy site to stop a blood leakage.

The connecting fiber (105) passes through the plug (100) and through a frame connection (125) located on the plug frame (115); the frame connection (125) can be a slidable or adjustable connection as shown in FIG. 3A where the connecting fiber (105) passes through the plug frame (115). One end of the adjustable connecting fiber (105) is connected to a central hump (127) of an anchor (130) at the anchor attachment (132) and the other end extends through a cinch ring (135) and through a push member (140). A portion of the anchor (130) can extend beyond the distal end (10) of the inner sheath (95); another portion of the anchor (130) can be positioned on the outer surface (55) of the inner sheath (95) as shown in FIG. 3A. Alternately, the entire anchor (130) can be located within the inner sheath (95) and the inner sheath (95) can have an opening (17) at its distal end (10) similar to those described in FIGS. 1A-2C for the introducer sheath (5).

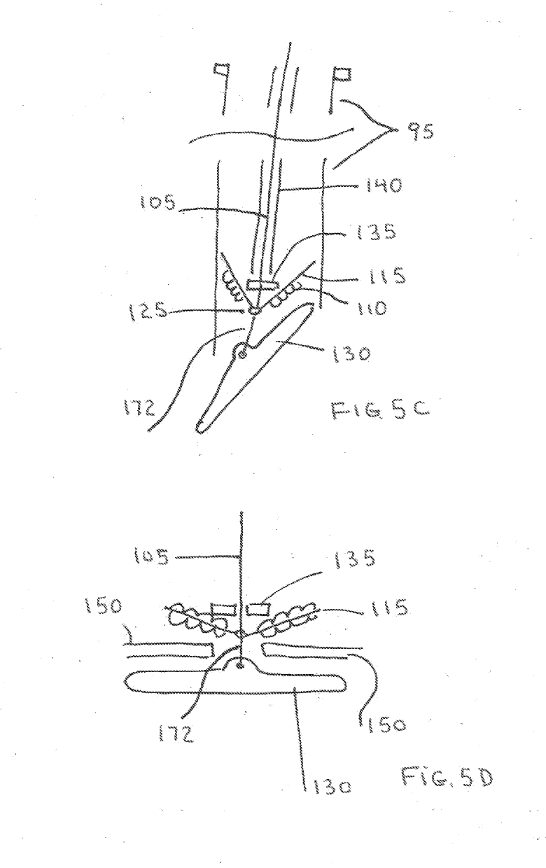

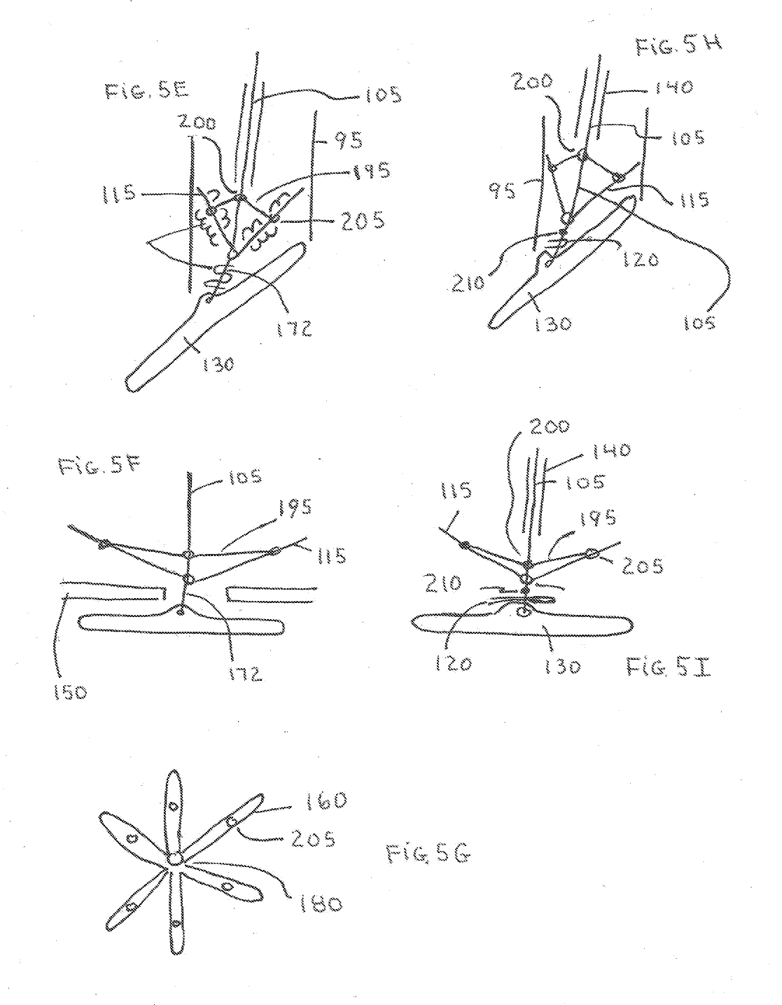

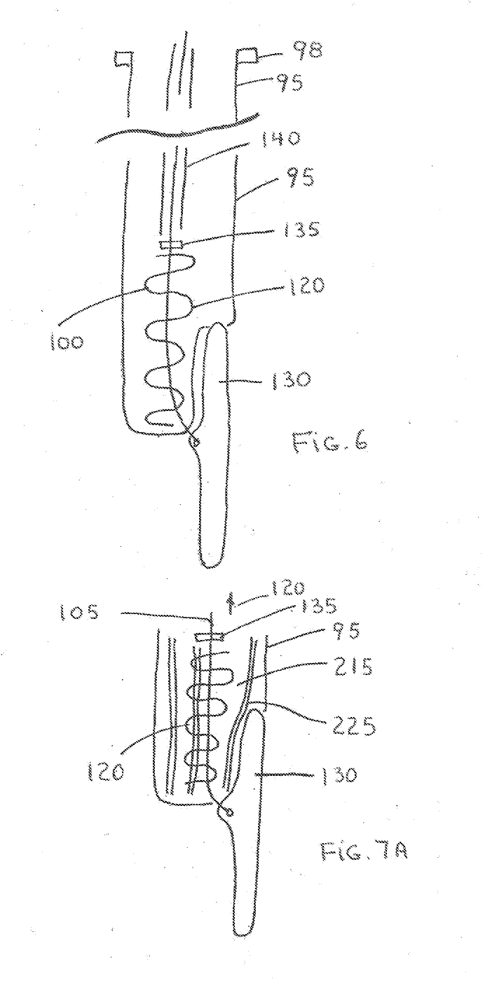

As the push member (140) is advanced over the connecting fiber (105) the plug (100) is advanced toward the anchor (130) until the plug (100) comes into contact with the inner surface (145) of the blood vessel wall (150) as shown in FIG. 3B thereby making the connecting fiber (105) adjustable in length. A cinch ring (135) provides a friction fit with the connecting fiber (105) to hold the plug (100) in a position against the outer surface (155) of the blood vessel wall (150). The cinch ring (135) also pushes the spokes (160) of the plug frame (115) outwards (see FIGS. 5A and 5B) or pushes other portions of the plug (100) outward to ensure that the plug (100) is not able to fit into the access site opening (165) in the vessel wall and can hold the anchor (130) tightly against the luminal side of the vessel wall. The frame connection (125) of the plug frame (115) can be formed with a friction fit with the connecting fiber (105) such that the cinch ring (135) is not necessary to hold the plug (100) tightly against the anchor (130), instead the frame connection (125) can slide downward to form a tight fit between the plug (100) and the anchor (130) but will not move outwards in the other direction, away from the anchor (130). The blood vessel wall (150) is thereby contained between the plug (100) and the anchor (130); the anchor holding the plug (100) tightly against the outer surface (155) of the blood vessel to provide a hemostatic seal of the vascular access site opening (165).

The plug (100) of one embodiment (shown in FIGS. 3A and 3B) has a frame that is formed of an elastic biodegradable material such as PGA, PLA, TPC, or copolymer or these, or mixture of these or other biodegradable polymers having an elastic character such that it will retain a significant amount of its shape over a period of at least several hours while in the body under stress. The purpose of the frame is to expand out due to stored elastic energy from its collapsed configuration as it is held by the inner sheath (95) to a larger diameter that is larger than the diameter of the access site opening (165). The frame does not allow the plug (100) to extend through the access site opening (165) into the vessel lumen where it can embolize into the blood stream.

The cinch ring (135) can be formed from a biodegradable material similar to those described for the plug frame (115) or the anchor (130). The cinch ring (135) has a center hole that allows for passage of a biodegradable connecting fiber (105). The center hole has a tortuous path in the form of a zig zag with sharp corners to the zig zag directed away from the plug (100). Upon advancing the cinch ring (135) toward the plug (100), the cinch ring (135) forms a friction fit that does not allow it to move away from the plug (100), thereby holding the plug (100) firmly against the anchor (130) or against the outside surface of the blood vessel wall (150). A slip knot can also be used to hold the plug (100) securely down against the outside surface of the blood vessel wall (150). The connecting fiber (105) can be cut using mechanical or thermal mechanism after the plug (100) has been secured relative to the anchor (130).

The soft plug portion (110 or 120) of one embodiment is formed from one or more biodegradable materials that can expand upon exposure to blood and are compressible. The soft plug portion can be formed from porous, particulate, fibrous biodegradable material, or foam biodegradable collagen or gelatin, or other biodegradable materials such as PEG; the plug (100) can contain thrombogenic agents or materials such as starches known to cause thrombosis, and can contain water-swelling materials such as hydrogels. An upper soft plug portion (110) is attached to the plug frame (115) as shown in FIG. 3A. A lower soft plug portion (120) can be attached to the plug frame (115), attached to the upper soft plug portion (110), or can be unattached and located between the plug frame (115) and the anchor (130) as shown in FIGS. 3A and 3B. The lower soft plug portion (120) is generally formed into a zig zag configuration and a series of folds (170) with the adjustable connecting fiber (105) extending across the zig zags or folds (170). This pattern for the lower soft plug portion (120) allows this plug portion to extend outwards to form a larger diameter and provide improved hemostasis at the site of the access site opening (165).

The connecting fiber (105) can be formed from a nonbiodegradable material such as Dacron or from a biodegradable material such as found in biodegradable sutures used in surgery. The fiber can also be formed from PGA, PLA, copolymers of these including polycaprolactone, and other biodegradable fiber materials. The biodegradable connecting fiber (105) must be capable of holding stresses between the anchor (130) and the plug (100) for periods of time until hemostasis has been established and stabilized, typically for a few days.

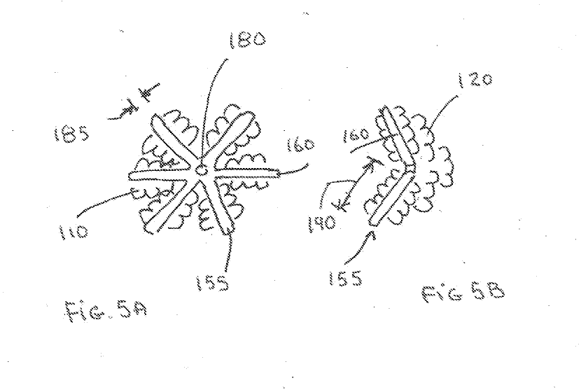

An alternate embodiment for an element of the present invention is shown in FIGS. 4A, 4B, 5A, and 5B. The construction materials for the plug (100), connecting fiber (105), and anchor (130) are similar to that described in FIGS. 3A and 3B. The fixed connecting fiber (172) of this embodiment is not adjustable, but instead has been fixed to specific distance connecting the anchor (130) to a frame connection (125). The fixed connecting fiber length (175) can be approximately equal to the wall thickness of the blood vessel (approximately 0.5-1.5 mm) or a thickness that provides compressive stress between the plug (100) and the outer surface (155) of the vessel wall and the anchor (130) and the inner surface (145) of the blood vessel wall (150) (approximately 0-4 mm). The fixed length (175) of connecting fiber (172) can obviate the need for a cinch ring (135) or not to be formed in the connecting fiber to hold the plug (100) securely against the outside of the vessel wall. The fixed connecting fiber (172) can be made, for example, of an elastic material such as polyurethane, silicone, or a PET wound helically around an elastic polymer fiber. The modulus of the elastic fixed connecting fiber (172) should be large enough to hold the plug (100) and anchor (130) together without blood leakage from the vessel but low enough to allow the plug (100) to be pulled out of the inner sheath (95) during deployment of the plug (100).

The anchor (130) can be positioned at a bevel within the inner sheath (95) as shown in FIG. 4A or it can be positioned at a bevel with respect to the axis of the inner sheath (95) as shown in FIG. 3A. This beveling of the anchor (130) will allow the anchor (130) to fit between the distal end (10) of the introducer sheath (5) (FIG. 1A) and the wall of the blood vessel opposite to the vascular access site. The lower soft plug portion (120) between the plug frame (115) and the anchor (130) is required to swell and provide for hemostasis until the upper soft plug portion (110) approaches the outer surface (155) of the blood vessel wall (150) after release of the plug frame (115) from the inner sheath (95).

FIGS. 5A and 5B show the plug (100) in a relaxed state from a frontal view and from a side view, respectively. From the frontal view the frame has a frame opening (180) that provides passage for the connecting fiber (105) or for permanent attachment for the fixed length connecting fiber (172). The plug frame (115) has several spokes (160) that extend out radially and are able to bend upon placement into the inner sheath (95). The spoke width (185) can range from 0.003-0.010 inches. The spoke length (190) can range from approximately 3 mm to 8 mm. The upper soft plug portion (110) is located between each of the spokes (160) and also around the spokes (160). The lower plug portion is can be attached to the plug frame (115) as shown in FIG. 5B or it can be unattached and located between the plug frame (115) and the anchor (130).

As shown in FIGS. 5C and 5D, the connecting fiber (105) can further extend from its permanent or sliding attachment to the frame connection (125) through the entire inner sheath (95). The fixed connecting fiber (172) (as described in FIGS. 4A and 4B) can permanently join the plug frame (115) at the frame connection (125) with the anchor (130). In one embodiment a single connecting fiber (105) can extend through the inner sheath (95), can form a knot or fixed attachment to the plug frame (115) and can continue further to attach to the anchor (130). Alternately, the connecting fiber (105) can be releasably attached to the plug frame (115), for example at the frame connection (125). Such a releasable attachment can be made between the connecting fiber (105) and the plug frame (115) in the form of a double strand of the connecting fiber (105) that loops around the frame opening (180) in the plug frame (115); release of one end of the double strand will release the plug frame (115) from the connecting fiber (105) after the anchor (130) and plug (100) have been deployed. A fixed connecting fiber (172) can permanently attach the plug (100) with the anchor (130). The connecting fiber (105) that extends through the inner sheath (95) can be used to ensure that the anchor (130) is pulled snugly against the end of the inner sheath (95) or introducer sheath (5) after it has been appropriately delivered to the vessel lumen. Additionally, the connecting fiber (105) can be used to pull the anchor (130) tightly against the vessel wall as shown in FIG. 5D.

The embodiment of FIGS. 5C and 5D can also include a cinch ring (135). A cinch ring (135) can be placed over the connecting fiber (105) and can be pushed against the plug (100) using a push member (140). The cinch ring (135) can apply a force onto the plug frame (115) to force the spokes (160) outward to ensure that the plug (100) does not embolize into the blood vessel lumen (250) and to hold the anchor (130) securely against the luminal side of the blood vessel wall (150).

The embodiment of FIGS. 5E and 5F has an additional component to the plug frame (115), a plug frame lock (195). The connecting fiber (172) can have a fixed distance from the anchor (130) to the frame connection (125) as shown in FIG. 5E. The connecting fiber (105) can extend through the lock connection (200) such that it can slide with respect to the lock connection (200). The frame lock (195) is attached to the plug frame (115) along one or more of the frame spokes (160) in a pivotal connection (205). A push member (140) advances the lock connection (200) toward the frame connection (125) to push the spokes (160) of the frame outward into an expanded configuration as shown in FIGS. 5F and 5G. The plug frame (115) and frame lock (195) are held in position by using a toggle design that forces the lock connection (200) to extend over center in a manner similar to an umbrella. Alternately, the lock connection (200) can be formed with a slip fit or a friction fit that allows movement in only one direction such that it moves toward the plug (100) but not in the opposite direction. Alternately, a cinch ring (135) can be place over the safety connecting fiber (105) and can be advanced toward the anchor (130) using a push member (140) as described earlier.

In another embodiment shown in FIGS. 5H and 5I the plug (100) is slidably attached to both the frame connection (125) and the lock connection (200). A connecting fiber (105) attached to the anchor (130) extends through the frame connection (125) and the lock connection (200) and through a push member (140). Advancing the lock connection (200) toward the anchor (130) causes the frame lock (195) and plug frame (115) to move toward the plug (100) and form a friction fit between the lock connection (200) and the connecting fiber (105). The plug frame connection (125) can slide along the connecting fiber (105) until it comes into contact with a frame stop (210). The lower soft plug portion (120) located between the plug frame (115) and the anchor (130) is compressed as the plug frame (115) is moved toward the anchor (130) by movement of the push member (140). The distance between the anchor (130) and the plug (100) is adjustable in this embodiment. The connecting fiber (105) can be used to pull the anchor (130) tightly against the vessel wall and ensure that embolization does not occur; the entire vascular closure has been deployed against the vessel wall as shown in FIG. 5I.

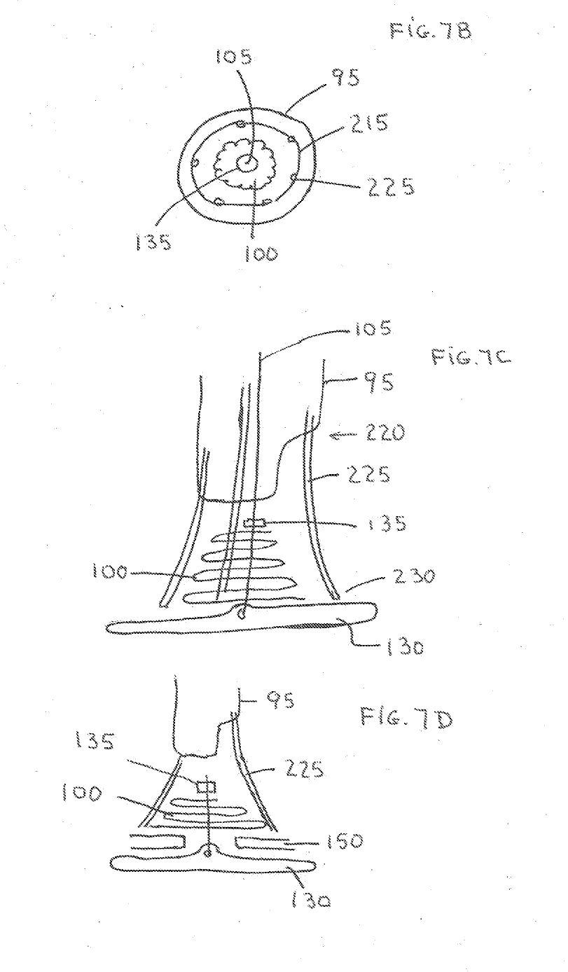

In an alternate embodiment, the plug (100) does not include a plug frame (115) and only contains a lower soft plug portion (120) as shown in FIG. 6. A push member (140) pushes the cinch ring (135) downward to compress the soft plug portion into contact with the outer surface (155) of the blood vessel wall (150).

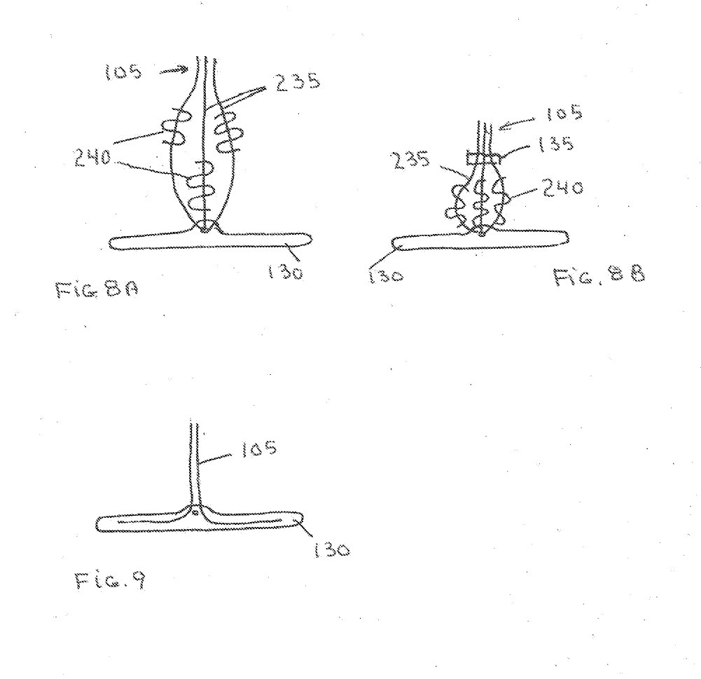

To improve the delivery of the soft plug portion to a position adjacent to the outer surface (155) of the blood vessel wall (150) an expandable plug container (215) surrounds the plug (100) as shown in FIGS. 7A-7D. As the soft plug portion is compressed by advancement of the cinch ring (135) toward the anchor (130), the expandable plug container (215) expands outwards at its distal end (230) to a diameter that is larger than the diameter of the inner sheath (95). This expansion allows the plug (100) to expand outward along the outer surface (155) of the vessel wall to a larger diameter than the access site opening (165). The proximal end (220) of the expandable plug container (215) does not expand thereby directing the plug (100) outwards as it travels toward the blood vessel wall (150). This expanded plug (100) diameter will limit the ability of the plug (100) to enter into the access site opening (165) and will obviate the possibility for embolization of the soft plug portion into the lumen of the blood vessel.

The expandable plug container (215) can have axial wires (225) or strands attached along a perimeter as shown in a cross-sectional view of FIG. 7B. The axial supports allow the expandable plug container (215) to retain its shape of a funnel (245) as the plug (100) is being advanced toward the blood vessel wall (150) near the (230) of the expandable plug container (215). The axial supports can be metal wires or polymer fibers that are bonded to a thin polymeric membrane that forms the shaped funnel (245) of the expandable plug container (215).

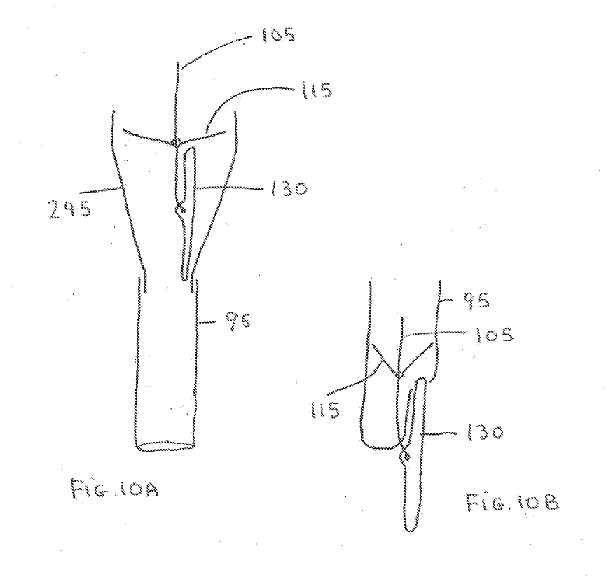

The connecting fiber (105) of the present invention can be made of more than one connecting filament (235), where each connecting filament (235) contains a plug segment (240). As shown in FIGS. 8A and 8B three individual plug segment (240) can each have a separate connecting filament (235) extending through it; each connecting filament (235) being attached to the anchor (130). The three connecting filaments (235) can each extend through the cinch ring (135) as shown in FIG. 8B. Upon advancement of the cinch ring (135) toward the anchor (130), each of the plug segments (240) can follow over a specific connecting filament (235) and thereby deliver the plug material individually to a region near the anchor (130) that forms a larger diameter and thereby provides both an improved seal for the access site opening (165) as well as provide a larger diameter plug (100) that will not embolize into the blood vessel lumen (250).

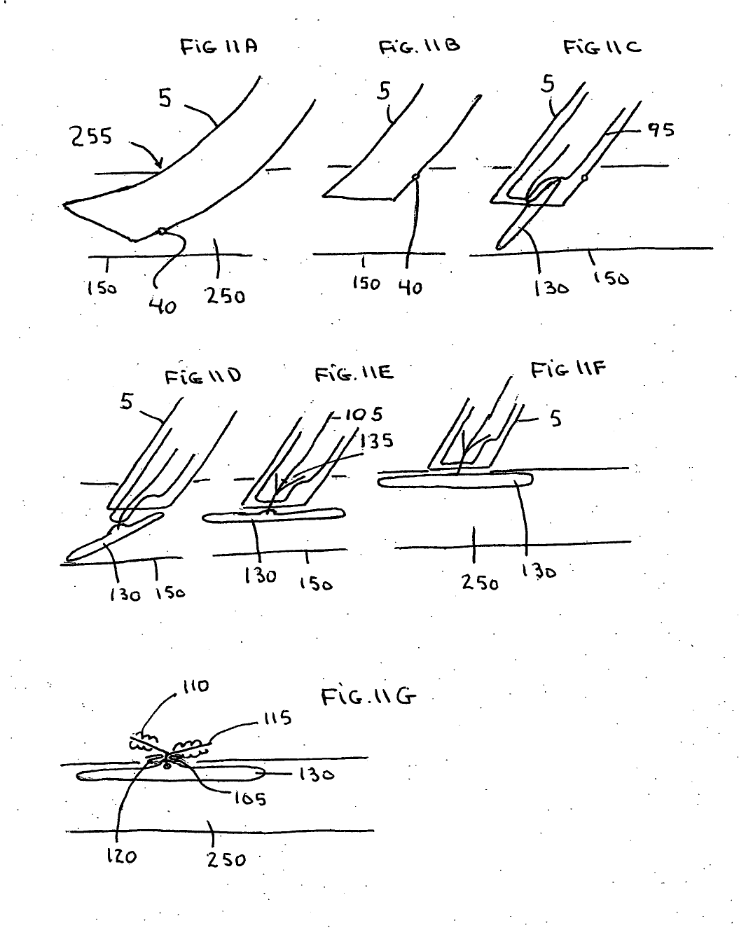

One or more connecting fibers (105) or connecting filaments (235) can extend into the anchor (130) as shown in FIG. 9. The presence of a connecting fiber (105) or connecting filament (235) inside the anchor (130) will provide the anchor (130) with a strength in extension that is often not found with many biodegradable materials such as PGA and PLA. The connecting fiber (105) can be formed from a material with a higher degree of tensile strength than PGA and PLA and thereby allow the anchor (130) to achieve a longer length as needed for sealing a larger diameter access site opening (165).

To ensure that the plug frame (115) does not deform to relieve its stress while it is placed within the inner sheath (95) under stress, it is intended that the frame be placed into the inner sheath (95) just prior to delivery to the blood vessel. The plug frame (115) is stored in a relaxed state as shown in FIG. 10A in a funnel (245) along with the anchor (130) and connecting fiber (105). Just prior to use, the anchor (130) and plug frame (115) are advanced through the funnel (245) to achieve a smaller diameter representative of the inner sheath (95) (see FIG. 10B) and is ready for delivery into the introducer sheath (5) and deployment to provide closure to the large diameter access site opening (165).

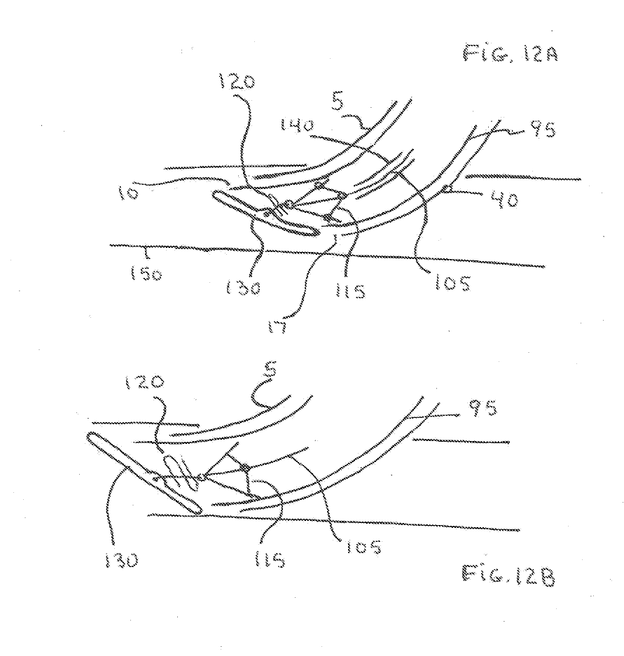

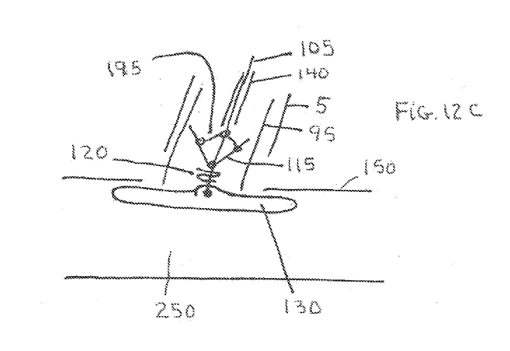

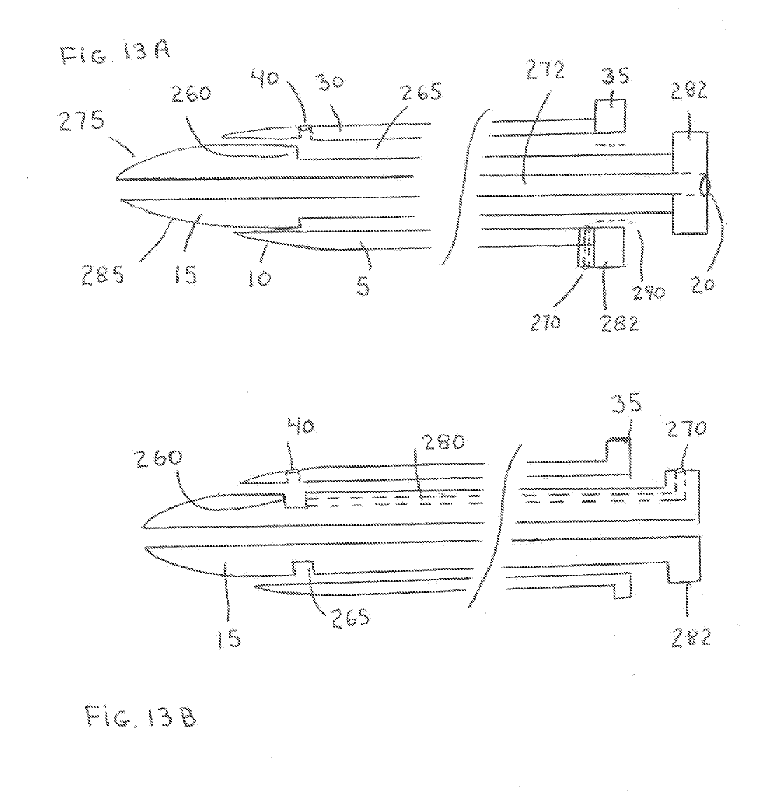

The method of use is shown in FIGS. 11A-G. The introducer sheath (5) is shown with the interventional therapeutic catheter removed in FIG. 11A and is positioned such that the weep hole (40) is located just inside the blood vessel lumen (250) across the arteriotomy site (255) as shown in FIG. 11B. The inner sheath (95) is advanced into the introducer sheath (5) and the anchor (130) is delivered into the vessel lumen between the distal end (10) of the introducer sheath (5) and the wall of the blood vessel opposite to the vessel access site opening (165) as seen in FIGS. 11D and 11E and pulled tight against the introducer sheath (5). The introducer sheath (5) and inner sheath (95) are pulled back to place the anchor (130) into contact with the inside of the blood vessel as shown in FIG. 11F.

For the embodiment shown in FIGS. 4A and 4B, the introducer sheath (5) and inner sheath (95) are withdrawn while holding tension onto the anchor (130) thereby releasing the plug frame (115) and soft plug portions.

For the embodiment shown in FIGS. 3A, 3B, and 6 the push member (140) is advanced to push a cinch ring (135) downwards to compress the lower soft plug portion (120) into contact with the outer surface (155) of the blood vessel wall (150). The introducer sheath (5) is pulled back along with the inner sheath (95) while tension is applied to the connecting fiber (105) to bring the plug (100) into closer contact with the outer surface (155) of the blood vessel wall (150). The connecting fiber (105) is cut with mechanical means or separated and sealed via thermal or melting means such as a heated electrical resistance wire.

For the embodiment shown in FIGS. 7A-7C, the introducer sheath (5) is retracted while tension is applied to the connecting fiber (105) and the push member (140) is advanced toward the blood vessel. The inner sheath (95) is retracted approximately 5-10 millimeters exposing the expandable plug container (215). The push member (140) is advanced further causing the plug (100) to expand the distal end (230) of the expandable plug container (215) depositing plug (100) material into contact with the outer surface (155) of the blood vessel in a large diameter plug (100). The expandable plug container (215) is then pulled back to release the plug (100) against the outside of the blood vessel wall (150). The connecting fiber (105) is cut adjacent to the cinch ring (135).

It is understood that each of the embodiments for elements of the present invention provide specific advantages as described in this patent disclosure. Each of the embodiments for elements of the present invention can be used interchangeably with other elements from other embodiments without deviating from the present invention. The reference numbers for each of the embodiments of the present invention describe components with similar description and function in other embodiments.

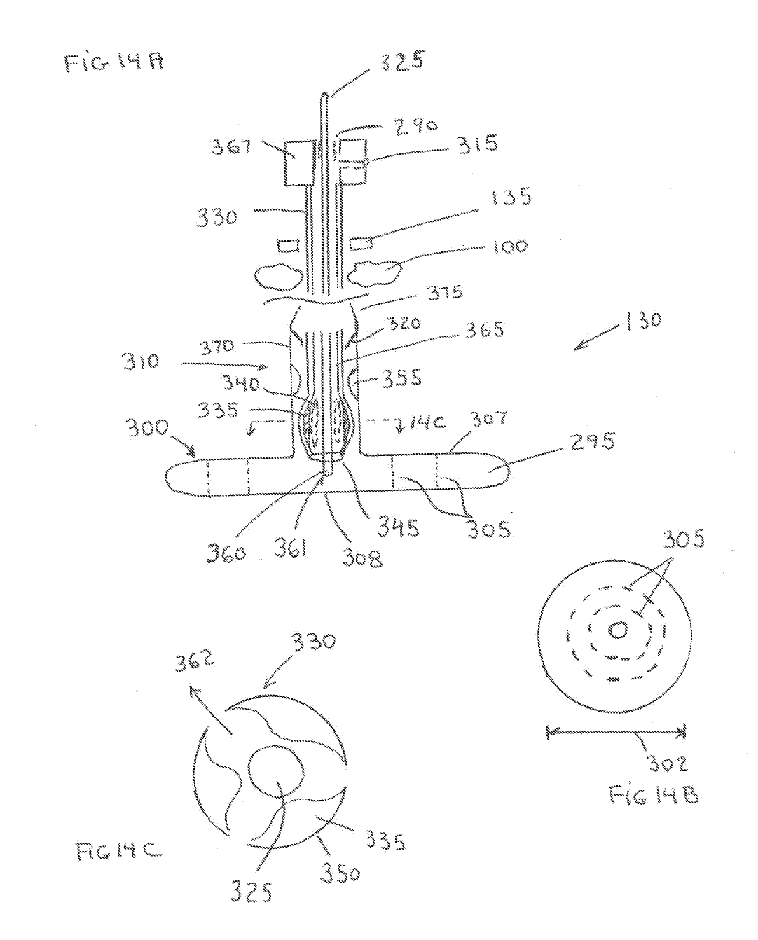

An alternate method of use is shown in FIGS. 12A-12D. In this method, the anchor (130) is deployed while the introducer sheath (5) is still significantly advanced into the blood vessel by a distance of approximately 0.8-1.5 cm (range of approximately 0.5-3.0 centimeters). The position of the introducer sheath (5) into the blood vessel lumen (250) has been identified by the position of the weep hole (40) at a safe distance into the blood vessel as shown in FIG. 12A. The inner sheath (95) is then positioned such that its distal end (10) extends beyond the distal opening (17) in the outer sheath. Using a push member (140), the anchor (130) is advanced out of the inner sheath (95) as shown in FIG. 12B. Tension is applied to the connecting fiber (105) to pull the anchor (130) into contact with the end of the inner sheath (95) as shown in FIG. 12C. The introducer sheath (5) and inner sheath (95) are withdrawn back until the anchor (130) has come into contact with the inner surface (145) of the vessel wall as shown in FIG. 12D. Further retraction of the inner sheath (95) and introducer are performed while applying tension to the connecting fiber (105) and pushing the push member (140) distally toward the anchor (130). The soft plug portion located between the anchor (130) and the plug frame (115) provides hemostasis and prevents a hematoma from forming. The anchor (130) will pull the plug frame (115) that has a fixed distance out of the inner sheath (95) and into position to plug the opening in the vascular access site. The frame lock (195) is advanced toward the anchor (130) by advancing the push member (140) toward the anchor (130); the spokes (160) of the plug frame (115) are pushed outward by the frame lock (195). Further advancement of the push member (140) will cause the frame lock (195) to move past center and hold the spokes (160) of the plug frame (115) outward to ensure that the plug (100) cannot embolize. The frame lock (195) can also have a friction fit with the connecting fiber (105) to further hold the lock connection (200) firmly with respect to the connecting member. Alternately, a cinch ring (135) can be placed over the connecting fiber (105) to assist in pushing the spokes (160) of the plug frame (115) outwards and ensure that a continual force is applied between the plug (100) and the anchor (130).

The method of use for an embodiment which provides a slidable and lockable frame is also anticipated, In this method of use, the plug frame (115) can be slid along the connecting fiber (105) by advancing the push member (140) until the plug frame (115) comes into contact with a frame stop (210). This advancement of the plug frame (115) causes soft plug portion located between the plug frame (115) and the anchor (130) to be compressed and placed into a compressed configuration into contact with the outside of the vessel wall. Further advancement of the push member (140) causes the frame lock (195) for form a toggle movement and lock the plug frame (115) into its final position as shown in FIG. 5I.

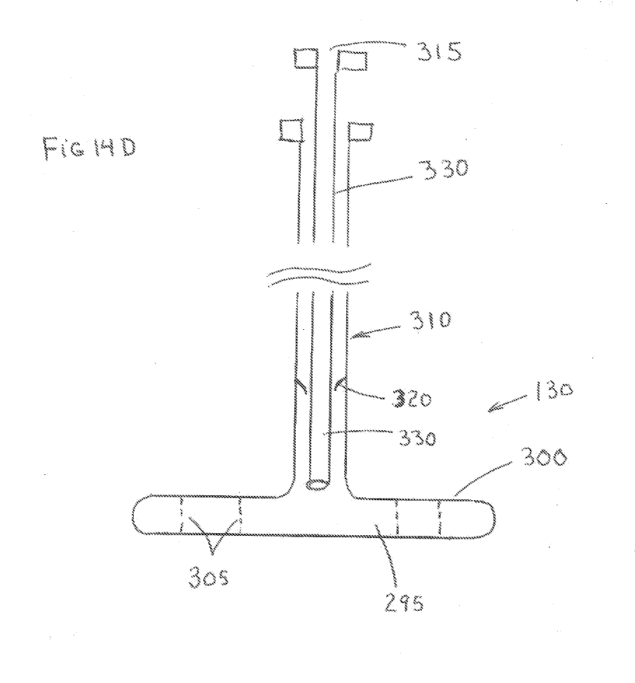

FIGS. 13A and 13B describe other embodiments for the introducer sheath (5) and dilator (15) that work together to allow positioning of the distal end (10) of the working introducer sheath (5) with respect to the arteriotomy site (255) of the blood vessel being accessed. The outside profile of the introducer sheath (5) is not affected by the ability of this introducer sheath (5) and dilator (15) to identify its position within the blood vessel with the weep hole being located slightly inside or slightly outside of the arteriotomy site. A small weep hole (40) ranging from 0.005 to 0.040 inches is formed through the wall of the introducer sheath (5) near the distal end (10) of the introducer sheath (5). The dilator (15) has a step (260) located in the dilator outer surface (285) proximal to the introducer sheath (5) distal end (10) that provides an annular space (265) for blood to flow into from the weep hole (40). The annular space (265) has a thickness that ranges from 0.003 to more than 0.040 inches. In FIG. 13A the annular space (265) extends proximally to the outer sheath manifold (35) where a weep fitting (270) is located to observe flow of blood from the blood vessel through the weep hole (40) through the annular space (265) and out of the weep fitting (270) when the weep hole (40) is positioned within the arterial blood vessel. If the weep hole (40) is located outside the blood vessel, then no blood flow is seen at the weep fitting (270). A guidewire lumen (272) extends from the dilator distal end (275) through the dilator manifold (282). In FIG. 13B the annular space (265) is located only near the of the dilator distal end (275) in a region adjacent to the location of the weep hole (40) in the introducer sheath (5). A separate weep lumen (280) is formed in the dilator (15) to provide blood flow to the weep fitting (270) located on the dilator manifold (282). The weep lumen (280) can extend within the wall (30) of the dilator (15) or along a portion of the dilator outer surface (285). A seal (290) such as a silicone hemostasis valve located on the introducer sheath (5) manifold (35) forms a fluid-tight seal (290) with the dilator (15) while still allowing for movement between the dilator (15) and the introducer sheath (5).

The method of use for the introducer sheath and the dilator of the embodiments of FIGS. 13A and 13B includes the placement of the introducer sheath (5) along with the dilator (15) into the blood vessel lumen using standard Seldinger methods. The dilator is removed and the therapeutic catheter is introduced into the blood vessel via the introducer sheath. Following the interventional procedure, the therapeutic catheter is removed and the dilator (15) is repositioned into the introducer sheath (5). The introducer sheath can then be retracted proximally out of the vessel until the weep hole (40) indicates that the distal end (10) of the introducer sheath (5) is close to the arteriotomy site within 0.5-2 cm, for example. The vascular closure device can then be introduced into the blood vessel lumen through the introducer sheath directly or via an inner sheath that fits within the introducer sheath. The introducer sheath can then be further retracted as the vascular closure device is being deployed.

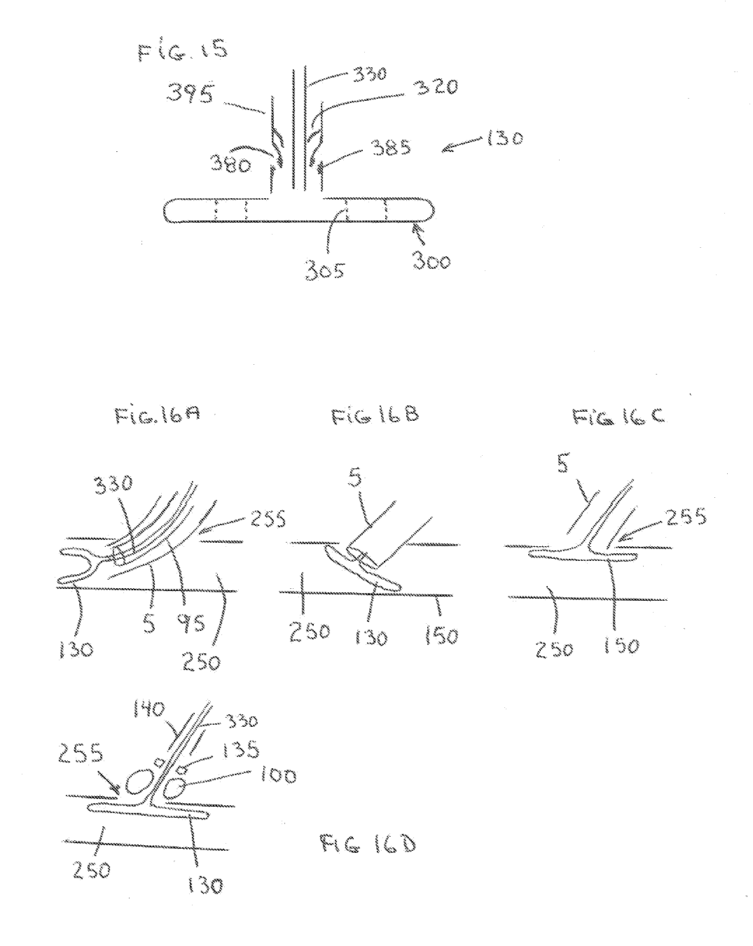

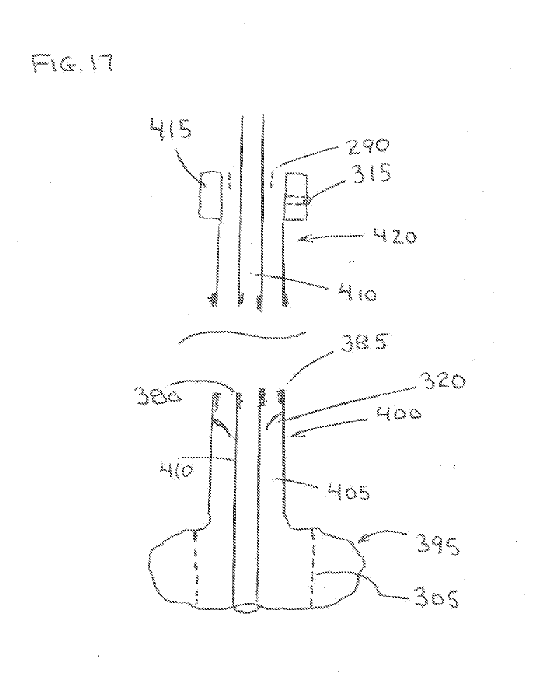

FIGS. 14A-15 show alternate embodiments for the anchor (130) that can be used with other embodiments of the plug (100) and introducer sheath (5) described throughout the present invention. The anchor (130) of these embodiments is intended to delivered in a smaller unfilled configuration and filled with a fluid medium (295) such as a polymerizable polymer or other fluid after it is delivered within the blood vessel. One advantage of such an anchor (130) is in closure of an arteriotomy site (255) for an introducer sheath (5) that is approximately the same diameter as the blood vessel diameter. When the introducer sheath (5) is being withdrawn back out of the arteriotomy site (255) following the therapeutic procedure, the anchor (130) of the present embodiments can expand out under the pressure of the inflating fluid (295) or polymeric fluid (295) to ensure that the anchor (130) remains within the blood vessel and cannot extend through the arteriotomy site (255) and out of the blood vessel.

FIGS. 14A-15 show an anchor (130) that has an anchor bag (300) that contains the fluid medium (295) or polymer or gel. The anchor bag (300) can have a flat pancake-type or disc shape with the thickness of the flat shape ranging from 0.5 to 2 mm and the anchor bag diameter (302) ranging from approximately 5-16 mm depending upon the diameter of the arteriotomy site (255). An arteriotomy diameter of 18 French (6 mm), for example, could have an anchor diameter of approximately 10-14 mm in order to help provide a seal of the arteriotomy site (255) from blood leakage. It is anticipated that the flat pancake shape would be maintained into the anchor bag (300) by holding fibers (305) that extend from the top surface (307) to the bottom surface (308) of the anchor bag (300). The anchor fibers (305) can be formed, for example, from a Dacron multifilament yarn or a single monofilament polymeric fiber that is sewn through each surface of the anchor bag to hold the surface into a close distance from each other. The anchor bag (300) can be made from woven materials such as Dacron, PTFE, ePTFE, or from a biodegradable fibers such as PLLA, PGA, form a composite of materials including fibers formed from metal fibers such as stainless steel or Nitinol combined with polymeric nondegradable or biodegradable fibers. The anchor bag (300) can also be formed from sheets of polymer such as sheets of ePTFE or an electrostatically spun fiber mesh such as polyurethane or silicone spun over an appropriately formed flat or pancake shaped mandrel or mold. Thermal heating processes can also be used to form the shape of the anchor bag (300) out of thermoplastic materials such as polyester, polyethylene, FEP, Teflon, and others used in medical device implants. The anchor bag (300) can be made porous such that during inflation with polymer fluid (295), the air (or possibly other gas such as carbon dioxide, if it is prefilled with this fluid (295)) or liquid such as saline, or other fluid (295) contained within the bag is extruded out of the porous material prior to filling with the polymer. The polymerizable polymer inflation fluid would be too viscous and would not pass through the pores of the anchor bag. Material surface energy differences between the anchor bag (300) and the polymeric inflation fluid can also prohibit passage of the polymer through the pores. The pore size for an ePTFE anchor bag (300), for example, could have an internodal distance ranging from 3-60 microns and could preferentially range from 5-30 micron internodal. Woven materials or spun materials used for the anchor bag (300) could, for example, be formed with spacing between fibers that range from 1-30 microns depending upon the viscosity of the filling fluid (295) that is to be contained within the anchor bag (300) and the viscosity of the fluid (295) that is expected to penetrate through the anchor bag (300). Alternately, the anchor bag (300) can be coated with an elastic polymer such as silicone or polyurethane such that it can be filled with low viscosity fluids including saline or a gas such as carbon dioxide either permanently or as a preliminary fill prior to replacement of the filling fluid (295) with a polymerizable polymer.

The anchor bag (300) can be filled with a polymerizable polymer such as two-part polyurethane or silicone that is used commonly in the medical device industry and can be either water soluble or non-water soluble and the polymer can be biodegradable or alternately be resistant to biodegradation. Solvents used during the polymerization should not cause an adverse reaction with the body, hence making water soluble systems attractive. Also, if saline is first used to fill the anchor bag (300), then compatible water soluble polymer systems would be used. Other polymer systems that are included in the present invention include two and three component systems that contain one or more of the chemicals including moieties, copolymers, or various forms of polyethylene glycol, mercaptopropionate, glycine, ethylene vinyl alcohol, polyacrylamide, UV-curable acrylic polymers, and other materials used for fluid introduction into a medical device found in the human body.

The anchor bag (300) is attached to an anchor stem (310) which can be contiguous with the anchor bag (300). The anchor bag (300) and anchor stem (310) is delivered to the vascular access site and then is detached. A fill tube (330) delivers the polymeric fluid (295) from the fill hole (315) outside the body to the anchor bag (300) deployed to the inside of the vessel lumen. A valve (320) located in the anchor stem (310) prevents the polymer from escaping from the anchor stem (310) and maintaining the anchor bag (300) in an inflated configuration. The valve (320) can be a polymeric bileaflet valve (320) or duck-bill valve (320) with two flexible polymeric leaflets formed from polyurethane or other polymeric material. Once the inflation fluid (295) or polymer is contained in the anchor bag (300), it can be polymerized to form a solid or elastic solid or can be retained as a fluid (295) or a gel. This polymerization can occur in a few minutes and can range for up to an hour to obtain substantial polymerization. Polymerization can be enhanced by application of UV energy, for example, to the site of the anchor bag (300) to initiate the polymerization reaction. The shape of the polymerized polymer contained in the anchor bag (300) will conform to the shape of the blood vessel and should form a shape similar to a saddle.

One embodiment for delivery of the polymer fluid (295) to the anchor bag (300) is shown in FIGS. 14A and 14C. A mandrel (325) is placed through the fill tube (330) which has ridges (335) and slots (340) near its distal end (345) contained in the anchor stem (310). The ridges are formed from a polymeric material, for example, and are attached or formed onto the inside surface of the fill tube (330) at, for example, three discrete locations along the perimeter of the fill tube (330). The mandrel (325) impacts upon the ridges (335) causing the slots (340) to open up and place the outside surface (345) of the ridges (335) into contact with a stop (355) that is fixedly attached to the anchor stem (310). The slots are cuts that are made in the wall of the fill tube at locations between the ridges and allow the fill tube to expand in diameter at the location of the slots. The anchor stem (310) is generally smaller in diameter than the anchor bag and can be made of similar materials as the anchor bag and can be contiguous with it. Pulling proximally on the fill tube (330) allows the anchor (130) to be pulled against the inner wall of the blood vessel against the arteriotomy site (255) snugly to form a seal to reduce or prevent blood leakage out of the arteriotomy site (255). The mandrel (325) can have a UV probe (360) attached via a fiber optic at its distal end (361) or other polymer curing means such as heat or energy to help initiate polymerization of the polymeric fluid (295). Polymeric fluid (295) injected into the fill hole (315) can travel with a polymer path (362) down the annular region (365) of the fill tube (330), out of the slots (340), and into the anchor bag (300). A seal (290) can be located in the manifold (367) to prevent leakage of polymeric fluid (295). A cinch ring (135) can be placed over the fill tube (330) and can lock via friction with the outer surface (370) of the anchor stem (310) or filling tuber which effectively becomes a connecting fiber (105) in a manner similar to that described by previous embodiments. A push member (140) can advance the plug (100) over the tapered end (375) of the anchor stem (310); a cinch ring (135) or suture knot can hold the plug (100) tightly against the anchor (130). Withdrawal of the mandrel (325) will allow removal of the fill tube (330) thereby leaving the anchor (130) along with a plug (100) in place to provide hemostasis of the arteriotomy site (255).

FIG. 14D and 15 show other embodiments for a fill tube (330) contained within an anchor stem (310) having a valve (320) to prevent leakage of the polymeric fluid (295). Other materials for construction of this anchor (130) and polymeric fluid (295) are similar to that describe in FIG. 14A. The anchor stem (310) of this embodiment is pulled under tension proximally to place the anchor bag (300) into contact with the luminal side of the blood vessel at the arteriotomy site (255) following filling with a polymeric fluid (295) from the fill hole (315). Upon remove of the fill tube (330), a plug (100) and cinch ring (135) (or knot) can be applied to hold a plug (100) tightly against the anchor (130) in a manner similar to that described in FIG. 14A or earlier embodiments. For example, the fill tube (330) can be a braided or polymeric flexible tube that functions as a connecting fiber (105) to hold the plug (100) to the anchor (130). The anchor stem (310) can be cut using mechanical means or via thermal means such that it does not extend or protrude through the skin of the patient. Alternately, as shown in FIG. 15 the anchor stem (310) can have an inner thread (380) and outer thread (385) such that the proximal anchor segment (390) can be easily detached from the anchor stem (310) following polymeric fluid (295) delivery and placement of a plug (100). Reference numerals presented in the embodiments of the present invention can be applied to other embodiments of the invention.

FIG. 16 shows the method of operation for the placement of the embodiment shown in FIGS. 14A-15. A working introducer sheath (5) is placed into the blood vessel at the arteriotomy site (255). The introducer sheath (5) is withdrawn such that its distal end (10) is near the arteriotomy site (255) within 0.5-2 cm if possible using the introducer sheath (5) presented in the present invention if possible. Alternately any introducer sheath (5) can be used. An inner sheath (95) containing the anchor (130) is advanced into the working introducer sheath (5) and into the blood vessel. The anchor (130) is advanced out of the inner sheath (95) and into the blood vessel where it is inflated to a pressure ranging from 0.5 atm to 5 atm such that it has a force acting to push it outwards into a flat shape but not so large a force that it restricts movement within the blood vessel or causes emboli to form due to scraping of plaque from the blood vessel wall (150) as shown in FIG. 16A. The working introducer sheath (5) along with the inner sheath (95) are withdrawn and the heel (75) of the anchor (130) moves into the space between the introducer sheath (5) and the vessel wall as shown in FIG. 16B. Further retraction of the introducer sheath (5) places the anchor (130) into contact with the arteriotomy site (255) as shown in Fig. C. The pressure in the anchor (130) is maintained while traction is provided on the anchor stem (310) for form the anchor (130) into a saddle shape while the polymer contained within it is polymerizing. A plug (100) and holding means such as a cinch ring (135) are advanced tightly against the anchor (130) to provide hemostasis from the outside of the vessel as shown in FIG. 16D. The anchor (130) is then detached from the anchor proximal region via means described in the various embodiments.

FIG. 17 shows an embodiment for a plug (100) that is formed from a plug bag (395) and a plug stem (400) that is similarly filled with a polymerized polymer after it is delivered generally to its site outside of the arteriotomy site (255). The plug bag (395) is formed from similar materials as described for the anchor bag (300); the plug stem can be contiguous with the plug bag but with a smaller diameter than the plug bag. The polymeric fluid (295) delivered to the plug bag (395) is similar to that described earlier for filling the anchor bag (300). The plug bag (395) can have holding fibers (305) to form it into a shape that is more flat than a spherical bag and thereby allow it to extend outwards to generate friction with the tract hole extending through the skin and allow it to lay in close proximity to the outside of the vessel at the arteriotomy site (255). The plug bag (395) has a valve (320) to prevent leakage of polymeric fluid (295) out of the fill passage (405) of the plug stem (400). This valve (320) can be constructed in a manner similar to the valve (320) in the anchor stem described earlier. A central passage tube (410) extends through the plug (100) if desired to provide for passage of a connecting fiber (105) that connects to an anchor (130) or hollow tube that connects to a fillable anchor bag (300) as described in earlier embodiments. The polymeric fluid (295) is introduced into the fill hole (315) in the plug proximal region (420); it travels down the fill passage (405) and into the plug bag (395). A seal (290) located at the plug manifold (415) prevents leakage between the central passage tube (410) and the plug proximal region (420). The plug proximal region (420) can be separated from the plug stem (400) via a threaded connection having an inner thread (380) and outer thread (385).

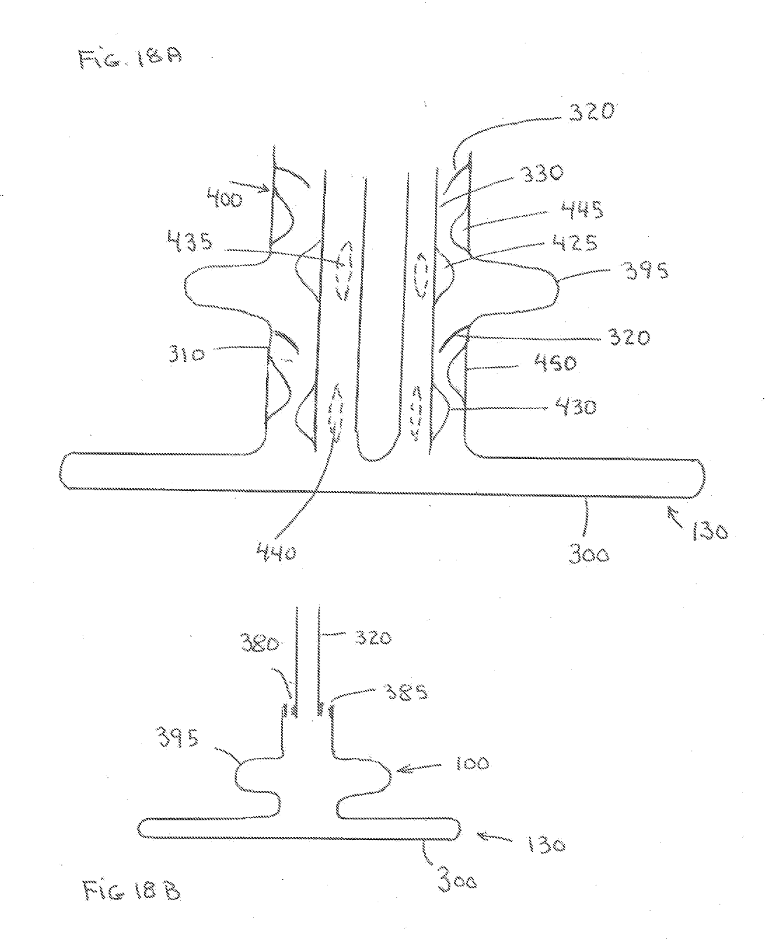

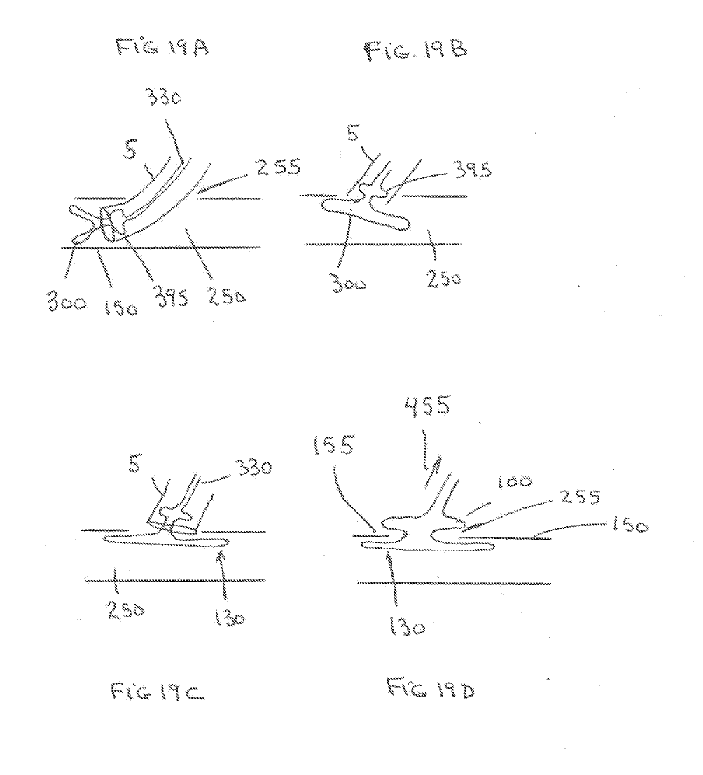

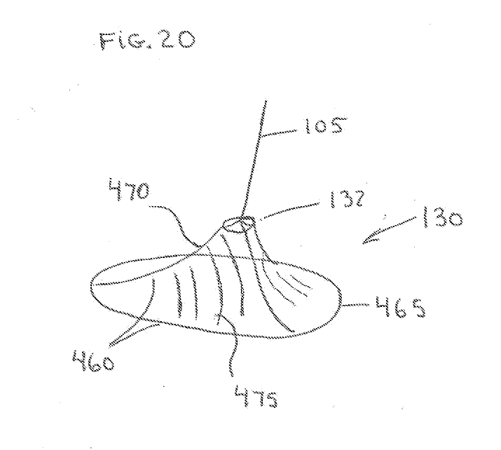

An alternate embodiment having the plug (100) and anchor (130) filled via a single fill tube (330) is shown in FIGS. 18A and 18B. The anchor bag (300) and plug bag (395) are formed from materials similar to those described earlier in the embodiments of FIGS. 14A to 17 and can be contiguous with each other. In the embodiment of FIG. 18A the anchor-plug has a fill tube (330) that has two regions with first ridges (425) and second ridges (430) and first slots (435) and second slots (440); the ridges (335) and slots function in a manner described in FIG. 14A. A mandrel (325) introduced into the fill tube (330) causes the ridges (335) located on the inside of the fill tube (330) to be pushed outwards thereby opening the slots (340) for flow of polymeric fluid (295) into the anchor (130) and the plug (100). The first ridges (425) and second ridges (430) also impact onto the first stop (445) and second stop (450), respectively, to prevent the plug stem (400) and anchor stem (310) from moving with respect to the fill tube (330) and thereby allowing traction of the fill tube (330) to pull the anchor (130) into place against the inner luminal surface of the blood vessel at the arteriotomy site (255). The fill tube (330) can be detached from plug stem (400) using inner and outer threads (385) as shown in FIG. 18B. Both the anchor (130) bag and plug bag (395) can be filled simultaneously in this embodiment if desired.

An alternate to the embodiment shown in FIG. 18A has only the second ridge and the second slots (440) and not the first ridge or first slots (435). For this embodiment the fill tube (330) is advanced to place the second ridges (430) just distal or adjacent to the second stops (450) and the mandrel (325) is in place across the second ridges (430) to hold the anchor stem (310) in place fixedly with respect to the fill tube (330) while polymeric fluid (295) is placed into the anchor bag first, before entering the plug bag (395). The anchor bag is placed into position against the arteriotomy site (255) by applying traction to the fill tube (330). Then the mandrel (325) is retracted or moved a small distance ranging from 0.5-2 cm and the fill tube (330) is then retracted and similarly moved a distance to place the second ridges (430) and second slots (440) just distal and into contact with the first stop (445). The polymeric fluid (295) is the delivered to the entire volume including the anchor bag and the plug bag (395). A valve (320) is attached to the plug stem (400) to prevent fluid (295) from escaping out of the plug stem (400). As second valve (320) can also be attached to the anchor stem (310) to ensure that fluid (295) pressure is maintained within the plug bag (395) during retraction of the mandrel (325) and the fill tube (330).

FIGS. 19A-D show the method of use for the embodiment of FIGS. 18A and 18B. The anchor (130) and plug (100) are delivered through either the introducer sheath (5) or the inner sheath (95) into the blood vessel near the site of the arteriotomy using the weep hole (40). The anchor bag is advanced out of the sheath and into the vessel lumen. The polymeric fluid (295) is introduced into the anchor bag causing it to push outwards against the vessel wall. Upon retraction of the introducer sheath (5) and vascular closure device (90) in a proximal direction (455), the anchor bag opens up to lock onto the arteriotomy site (255) as shown in FIG. 19B. Further retraction causes the anchor bag to seal against the arteriotomy site (255) as shown in FIG. 19C. Retraction of the mandrel (325) and fill tube (330) in a proximal direction (455) allow the plug bag (395) to be filled with polymeric fluid (295). A valve (320) between the plug (100) and the anchor (130) prevents polymer from escaping from the anchor bag and maintains its pressure within the anchor bag. Additional inflation of the plug bag (395) with inflation fluid (295) or polymeric fluid (295) at this time causes the plug bag (395) to inflate and create a seal around the blood vessel outer surface (155) as shown in FIG. 19D. Application of UV energy via a central mandrel (325) initiates curing of polymer to cure in preferentially 1-10 minutes to form a solid polymer. The polymer can be similar to those used, for example, in the dental industry for forming impressions or molded shapes of teeth structures.

FIG. 20 shows yet another embodiment for an anchor (130) that can be used with any plug (100) of the present invention to form a vascular closure device (90). This anchor (130) is comprised of an anchor frame (460) that can be formed preferentially from Nitinol wire or other elastic metal wire, fiber, or can be formed from metal processing methods including laser processing. The anchor frame (460) can also be formed from stainless steel wire or from a biodegradable material as described in earlier embodiments for the plug frame (115). The anchor frame (460) can be formed from a fiber having a diameter that ranges from 0.003-0.020 inches. The anchor frame (460) has a round shaped frame ring (465) that conforms to the inner surface (145) of the blood vessel when it is deployed forming an oval or saddle shape. During delivery the frame is folded or wound up or compressed to form as smaller profile that would fit within an inner sheath (95) having a diameter of approximately 16F-21F. Frame struts (470) connect the frame ring (465) to an anchor attachment (132) where the connecting fiber (105) is attached. The anchor frame (460) can have an anchor cover (475) attached along the perimeter of the anchor ring to assist in providing hemostasis at the arteriotomy site (255). The anchor cover (475) can be formed from Dacron, polyurethane, silicone, or other thin polymeric material in a woven or polymeric cast form and can be biodegradable and formed from materials such as PEG, PLLA, PGA, and others.

This embodiment for an anchor (130) is delivered in an introducer sheath (5) or inner sheath (95) into the blood vessel where it is deployed outwards and into the lumen of the blood vessel. As the introducer sheath (5) is retracted proximally, the anchor (130) is placed into contact with the arteriotomy site (255) conforming to the vessel wall and forming an oval or saddle shape. A plug (100) as described in any of the embodiments of the present invention, and which is either fixedly or slidingly attached to the anchor (130), is then delivered to the outside surface of the blood vessel as described in earlier embodiments.

It is understood that each of the embodiments of the present invention can be applied to other embodiments such that, for example, the plug (100) of one embodiment can be used with the anchor (130) of another embodiment. Further, the embodiments of the plug (100) and anchor (130) can be used with any of the embodiments of the introducer sheath (5) or dilator. Further, the reference numerals of each embodiment can be used to describe features found in alternate embodiments. The present embodiments are not limited to the drawings and specification as presented but are anticipated to extend beyond these embodiments.

1. A vascular closure device for closing an arteriotomy site in a blood vessel comprising;
A. an anchor located on the luminal side of the blood vessel, a plug located on the outer surface of the blood vessel, and a connecting fiber that connects said plug to said anchor, wherein;
B. said plug has a deliverable configuration smaller than the arteriotomy site diameter and a deployed diameter larger than the diameter of the arteriotomy site to provide hemostasis assistance to the arteriotomy site,
C. said anchor having a small deliverable diameter configuration, said anchor having an anchor bag with an anchor fill tube attached to said anchor bag, said anchor fill tube providing access for an inflation fluid that expands said anchor bag to a diameter larger than the arteriotomy site.

2. The vascular closure device of claim 1 wherein said inflation fluid is a polymerizable polymer.

3. The vascular closure device of claim 1 wherein said anchor has an anchor stem that contains a valve that prevents leakage of said inflation fluid out of said anchor bag through said anchor stem.

4. The vascular closure device of claim 1 wherein said anchor slides relative to said plug along said connecting fiber.

5. The vascular closure device of claim 4 wherein said connecting fiber is hollow said connecting fiber providing passage for said inflation fluid to said anchor bag.

6. The vascular closure device of claim 1 wherein said plug is formed from a biodegradable polymer.

7. The vascular closure device of claim 1 wherein said plug has a plug frame formed from a noncompliant material and a soft plug portion formed from a compliant material.

8. The vascular closure device of claim 1 wherein said plug comprises a plug bag having a plug fill tube attached thereto for said inflation fluid to enter into said plug bag and expand said plug bag from a diameter smaller than the arteriotomy site diameter to a diameter larger than the arteriotomy site diameter.

9. The vascular closure device of claim 8 wherein said plug bag has a plug stem with a valve to prevent flow of said inflation fluid out of said plug bag through said plug stem.

10. The vascular closure device of claim 9 wherein said inflation fluid is a polymerizable polymer.

11. The vascular closure device of claim 9 wherein said plug bag and said anchor bag are formed from a material that is porous to a gasseous material but prevents the flow of a polymeric inflation fluid to penetrate through it.

12. The vascular closure device of claim 9 wherein said plug bag and said anchor bag are formed from a material that extends contiguously from said anchor bag to said plug bag.

13. The vascular closure device of claim 12 wherein said anchor bag has an anchor fill tube that provides inflation fluid into said anchor bag and said plug bag has a plug fill tube that provides inflation fluid into said plug bag.

14. The vascular closure device of claim 9 wherein said anchor fill tube and said plug fill tube comprise a single anchor-plug fill tube that provides inflation fluid to both said anchor bag and said plug bag.

15. The vascular closure device of claim 14 wherein said single anchor-plug fill tube is movable to locate a distal end of said anchor-plug fill tube adjacent said anchor bag to fill said anchor bag without filling said plug bag, and locate a distal end of said anchor-plug fill tube adjacent said plug bag to fill said plug bag after said anchor bag has been filled.

16. The vascular closure device of claim 1 further comprising an introducer sheath and dilator, said introducer sheath providing passage for said dilator therethough, said introducer sheath having a weep hole near its distal end, said dilator having an annular ring located adjacent said weep hole, said annular ring having fluid communication with said weep hole and with a manifold of said dilator, said introducer sheath providing passage for said vascular closure device through the arteriotomy site with said introducer sheath weep hole positioned near said arteriotomy site.
